(19) United States
(12) Patent Application Publication
(10) Pub. No.: US 2008/0134417 A1
(43) Pub. Date: Jun. 12, 2008

(54) Goggles

(75) Inventor: Naoya Aoyama, Osaka (JP)

Correspondence Address: KODA & ANDROLIA, Suite 1140, 2029 Century Park East, Los Angeles, CA, 90067-2983, US

(73) Assignee: YAMAMOTO KOGAKU CO., LTD., Osaka (JP)

(21) Appl. No.: 11/579,964

(22) PCT Filed: May 12, 2005
(86) PCT No.: PCT/JP05/08685

§ 371 Date: Nov. 9, 2006

(30) Foreign Application Priority Data
May 13, 2004 (JP) 2004-143730

(51) Int. Cl. A61F 9/02

(52) U.S. Cl. 2/431; 2/426

(57) ABSTRACT

Goggles capable preventing fitting bodies (4) from being disconnected from bodies (2) against the intention of a user since hooks (3) are not easily separated from the bodies (2). The goggles comprise the bodies (2) and the fitting bodies (4) having the hooks (3) and connecting the hooks (3) to the outer end parts of the bodies (2). Vertical walls (7) rising roughly perpendicular to lens faces and pedestals (8) positioned roughly perpendicular to the vertical walls (7) and outwardly extending from the base parts of the vertical walls (7) are formed at the outer end parts of the bodies (2). Hook bodies (9) having hooking grooves (9a) facing toward the vertical walls (7) are projectedly formed on the pedestals (8). The hooks (3) form hooking holes (12) for the hook bodies (9), and hook parts (12a) to the hook grooves (9a) of the hook bodies (9) are formed at the inside parts of the hook holes (12).

### BACKGROUND OF THE INVENTION

1. Field of the Invention

The present invention relates to goggles, and more particularly, to goggles which are suitably used for sports such as swimming and skiing.

2. Description of the Related Art

As conventional goggles of this kind, there exists swimming goggles as shown in FIGS. 5 to 8. The swimming goggles 21 include glass bodies 22L and 22R, a flexible joint 23 for connecting and disconnecting inner ends of the glass bodies 22L and 22R, hooking tools 24 and a head-mounting member such as a head band 25. The hooking tools 24 are respectively attached to the opposite ends of the head band 25, and also detachably connected to the outer ends of the glass bodies 22L and 22R, so that the head band 25 forms a loop.

Each of the glass bodies 22L and 22R includes a receiving seat 26A which extends substantially perpendicularly to a lens surface, and a pedestal 26B which is integrally formed with a base portion of the receiving seat 26A. The pedestal 26B extends outward substantially perpendicularly to the receiving seat 26A. The glass bodies 22L and 22R also include a pair of to-be retained pieces 27 each having a to-be retained surface which is substantially in parallel to the receiving seat 26A. The receiving seat 26A located between the to-be retained pieces 27 is provided with a to-be engaged portion 28.

The hooking tool 24 is provided with an abutting seat 29 which is opposed to and comes into contact with the receiving seat 26A in a surface-to-surface relation. The abutting seat 29 includes an engaging portion 30 which can elastically engage and disengage with respect to the to-be engaged portion 28 in a male and female relation. The abutting seat 29 also includes retaining pieces 31 which engage with the to-be retained pieces 27 so as to prevent the hooking tool 24 from being disengaged in a longitudinal direction of the band with respect to the to-be retained pieces 27 and the pedestal 26B (see Japanese Patent No. 3199532 (Japanese Patent Application Laid-open No. 7-067983 (pages 2 and 3, FIGS. 2 to 5)).

According to the swimming goggles 21 having the above-described configuration, if the hooking tools 24 are attached to the glass bodies 22L and 22R from their front side, the retaining pieces 31 come to be located laterally inner sides of the to-be retained pieces 27, so that it prevents hooking tools 24 from being laterally outwardly detached from the glass bodies 22L and 22R. When the engaging portion 30 and the to-be engaged portion 28 elastically engage each other in the male and female relation, this prevents the hooking tools 24 from disengaging from the glass bodies 22L and 22R in the longitudinal direction. Further, upper and lower to-be retained pieces 27 are interposed between the abutting seat 29 which functions as a retaining piece and the upper and lower retaining pieces 31, thereby preventing the hooking tools 24 from being vertically detached from the glass bodies 22L and 22R.

As goggles of this kind, there is swimming goggles as shown in FIGS. 9 to 11. This swimming goggles 41 include lens portions 42 each having a front surface and a rear surface, frame portions 43 which surround the respective lens portions 42, and a head mounting member such as a head band 44 which is connected to rear ends of the frame portions 43.

Each of the frame portions 43 is provided at its rear ends with an upper arm 45 and a lower arm 46 extending backward parallel with each other. Both the arms 45 and 46 have projections 47 and 48 at their tip ends which the projections are facing each other and extending toward the other arm. A distance R between the projections 47 and 48 is smaller than a distance Q between both the arms 45 and 46 which are located anteriorly to the projections 47 and 48.

The head band 44 is attached to the frame portions 43 through connecting members 51 which have rubber elasticity and are detachably accommodated between both the arms 45 and 46. Each of the connecting members 51 includes a fixing portion 52, a flexible belt-like portion 53 extending rearward from a rear surface of the fixing portion 52 through between both the arms 45 and 46, and an insertion hole 54 for the head band 44 formed on the belt-like portion 53. The fixing portion 52 has a pair of side surfaces 55a and 55b facing to the upper arm 45 and the lower arm 46, and stopper projections 56 are respectively provided on the side surfaces 55a and 55b. The fixing portion 52 is accommodated from front in a recess 43a of the frame portion 43 between the upper arm 45 and the lower arm 46 so that a rear surface 52a of the fixing portion 52 is adhered to at least a part 57a of a smooth portion 57. The fixing portion 52 is also adhered to the projections 47 and 48 of the upper arm 45 and the lower arm 46 from the front. At least one of width and thickness of the belt-like portion 53 is set to be smaller than the distance R between the projections 47 and 48 (see Japanese Patent Application Laid-open No. 2003-62121 (pages 2 and 3, FIGS. 1 to 3)).

According to the swimming goggles 41 having the above-described configuration, when a user wears this goggles and the head band 44 is pulled downward as viewed from FIGS. 10 and 11, the fixing portion 52 of the connecting member 51 is strongly adhered to the projections 47 and 48 of the upper arm 45 and the lower arm 46 and the part 57a of the smooth portion 57 from the front, so that this prevents the fixing portion 52 from being pulled away rearward from between the arms 45 and 46. As shown in FIG. 11, the stopper projection 56 is located posteriorly to the upper arm 45 and the lower arm 46 to prevent the fixing portion 52 from coming off forward from the recess 43a.

### SUMMARY OF THE INVENTION

According to the swimming goggles 21 as the conventional goggles, since the hooking tool 24 is prevented from being disengaged with the weak engagement of the engaging portion 30 and the to-be engaged portion 28, when the hooking tool 24 receives a force for forwardly detaching the hooking tool 24 (a force acting in the direction of arrow B in FIG. 8), there is a possibility that the glass bodies 22L and 22R and the hooking tool 24 are separated, and therefore the head band 25 is come off from the glass bodies 22L and 22R. Further, in the case of the conventional swimming goggles 21, if the hooking tool 24 is made of soft material, the retaining force of the engaging portion 30 and the to-be engaged portion 28 decreases, and therefore the head band 25 tends to be come off easily from the glass bodies 22L and 22R. This is the reason why the hooking tool 24 must have been made of hard material mainly.

According to the swimming goggles 41 as the conventional goggles, if the swimming goggles 41 receives a force for pushing out the fixing portion 52 forward of the upper arm 45 and the lower arm 46 (force acting in the direction of arrow E in FIG. 10), there is a possibility that the engagements between the stopper projection 56 and the projections 47 and 48 of the upper arm 45 and the lower arm 46 are released, the frame portion 43 and the connecting member 51 are separated from each other, and the head band 44 is came off from the frame portion 43.

Hence, it is an object of the present invention to solve the problems of the conventional goggles, and it is an object of the invention to provide goggles in which the hooking tool (connecting member) between a glass body (frame portion) and a head band does not easily come off from the glass body (frame portion), and therefore the head mounting member such as the head band is not come off from the glass body (frame portion), even if the hooking tool (connecting member) receives forces from any directions, or even if the hooking tool (connecting member) is made of soft material.

Accordingly, the goggles of the present invention including a main body 2, connecting tools 3, and an attaching member 4 which is connected to outer ends of the main body 2 by the connecting tools 3, may be provided at the outer area of the main body 2 with walls 7 which extends substantially perpendicularly to a lens surface, pedestals 8 which extend outwardly from base portions of the walls 7 to be substantially perpendicular to the walls 7, and protrusions 9 formed on the pedestals 8 and being provided with connecting grooves 9a facing toward the walls 7, and may be provided on the connecting tools 3 with connecting holes 12 for inserting the protrusions 9 which are provided with connecting portions 12a being engaged with the connecting grooves 9a of the protrusions 9 on inner sides of the connecting holes 12.

Further, the goggles of the present invention including a main body 2, connecting tools 3, and an attaching member 4 which is connected to outer ends of the main body 2 by the connecting tools 3, may be provided at the outer area of the main body 2 with walls 7 which extends substantially perpendicularly to a lens surface, pedestals 8 which extend outwardly from base portions of the walls 7 to be substantially perpendicular to the walls 7, and protrusions 9 formed on the pedestals 8 and being provided with connecting grooves 9a facing toward the walls 7, and may be provided with an inclined wall 10 inclining from front to back on the inner side of the connecting tools 3, a hooking portion 11 for the attaching member 4 on the outer side of the connecting tools 3 and connecting holes 12 for inserting the protrusions 9 on the intermediate space of the connecting tools 3, which the connecting holes 12 are provided with connecting portions 12a being engaged with the connecting grooves 9a of the protrusions 9 on inner sides of the connecting holes 12.

Moreover, the goggles of the present invention including a main body 2, connecting tools 3, and an attaching member 4 which is connected to outer ends of the main body 2 by the connecting tools 3, may be provided at the outer area of the main body 2 with walls 7 which extends substantially perpendicularly to a lens surface, pedestals 8 which extend outwardly from base portions of the walls 7 to be substantially perpendicular to the walls 7, and protrusions 9 formed on the pedestals 8 and being provided with connecting grooves 9a facing toward the walls 7, and may be provided with an inclined wall 10 inclining from front to back on the inner side of the connecting tools 3, a hooking portion 11 for the attaching member 4 on the outer side of the connecting tools 3 and connecting holes 12 for inserting the protrusions 9 on the intermediate space of the connecting tools 3, which the connecting holes 12 are provided with connecting portions 12a for engaging with the connecting grooves 9a of the protrusions 9 on inner sides of the connecting holes 12. In this goggles, the pedestal 8 may be further provided at its upper and lower portions with fitting grooves 8a, and the inclined wall 10 may be further provided at its opposite ends with fitting portions 3a which are fitted into the fitting grooves 8a of the pedestal 8.

In the goggles of the present invention, the main body 2 may include left and right bodies 2L, 2R and a joint 5 which connects inner ends of the left and right bodies 2L, 2R. In the goggles of the present invention, the protrusions 9 of the main body 2 and the connecting holes 12 of the connecting tools 3 may respectively have streamlined shapes.

Since the goggles of the present invention has the above-described configuration, the connecting tool 3 of the main body 2 and the attaching member 4 does not easily come off from the main body 2, and therefore the main body does not come off from the attaching member 4, even if the connecting tool 3 receives forces from any directions and even when the connecting tool 3 is made of hard material. Accordingly, the goggles of the present invention are extremely convenient for a user.

Further, the goggles of the present invention in which the protrusions 9 of the main body 2 and the connecting holes 12 of the connecting tools 3 respectively have streamlined shape, are suitable for being used especially in sports activity since the streamline shape can reduce the water resistance of this goggles.

### BRIEF DESCRIPTION OF THE DRAWINGS

FIG. 1 is a partially exploded perspective view of the entire embodiment of the goggles of the present invention;

FIG. 2 is a partial enlarged perspective view of the goggles of the present invention showing a state where a connecting tool and one of main bodies are disconnected;

FIG. 3 is a sectional view of the goggles of the present invention showing the state where the connecting tool and the one of main bodies are disconnected;

FIG. 4 is a sectional view of the goggles of the present invention showing a state where the connecting tool and the one of main bodies are connected with each other;

FIG. 5 is an entire sectional view showing one example of conventional goggles;

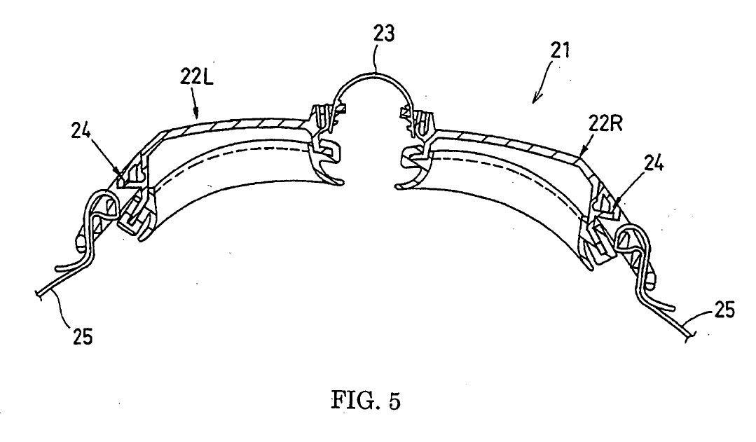

FIG. 6 is a plan view of the hooking tool and one of main bodies of the goggles shown in FIG. 5;

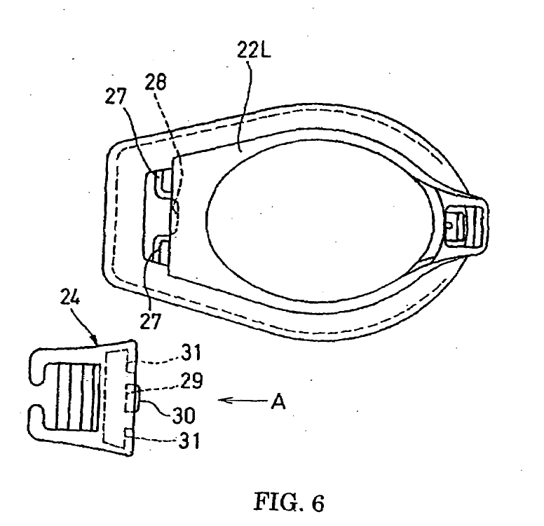

FIG. 7 is an explanatory diagram of the hooking tool as viewed from the direction of arrow A in FIG. 6;

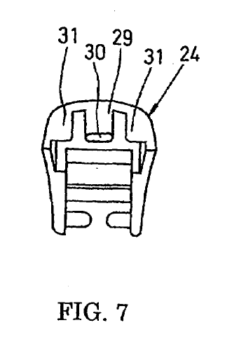

FIG. 8 is a sectional view of the goggles shown in FIG. 5 showing a state where the hooking tool and one of main bodies are disconnected;

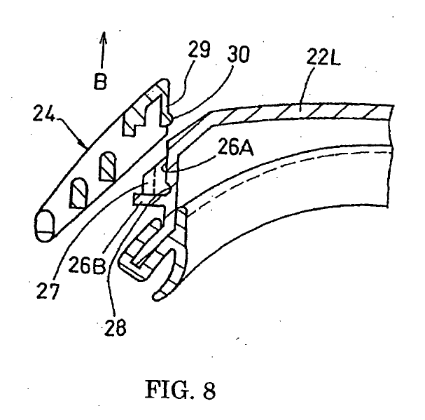

FIG. 9 is a partially exploded perspective view of another example of the conventional goggles;

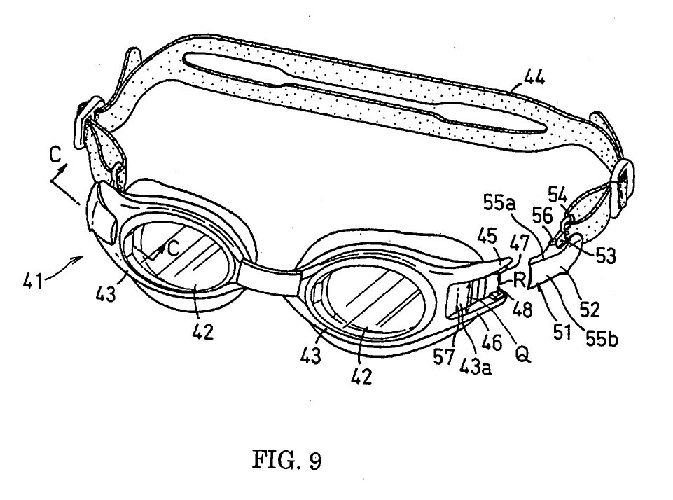

FIG. 10 is a sectional view taken along the line C-C in FIG. 9; and

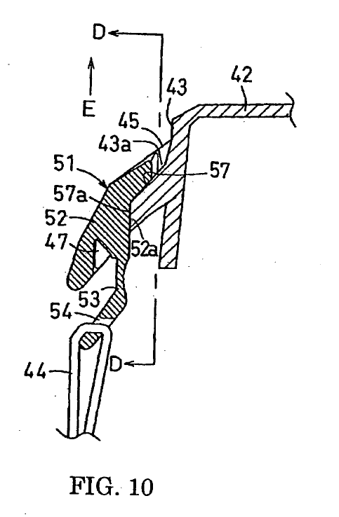

FIG. 11 is a sectional view taken along the line D-D in FIG. 10.

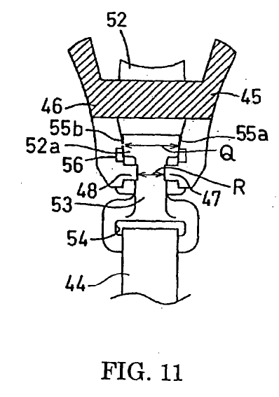

### DESCRIPTION OF THE PREFERRED EMBODIMENTS

The best mode of a goggle of the present invention will be explained in detail based on the drawings.

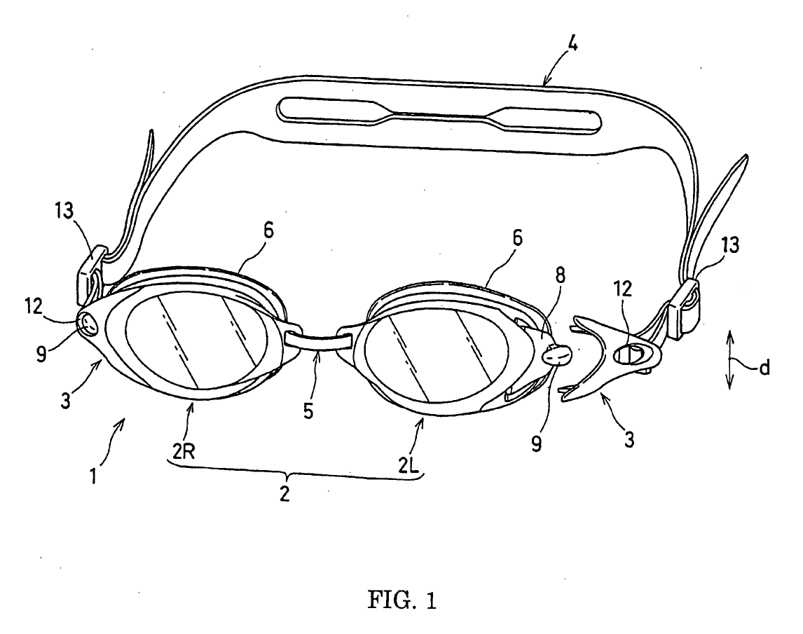

As shown in FIG. 1, the goggles 1 of the present invention include a main body 2, connecting tools 3 and an attaching member 4. The connecting tools 3, which are provided at the opposite ends of the attaching member 4, are respectively connected to outer ends of the main body 2. In FIG. 1, the main body 2 is described as a dual-lens type having left and right bodies 2L and 2R, but the main body 2 may be a single-lens type. The main body 2 is provided with a flexible joint 5 which detachably connects inner ends of the left and right bodies 2L and 2R in the case that the dual-lens type is selected as the main body 2, as shown in FIG. 1.

The main body 2 is made of transparent or semitransparent hard material such as hard synthetic resin. Lens surfaces thereof may have anti-fog finish if necessary. The left and right bodies 2L, 2R are provided with annular pads 6 on its side coming into contact with a face of a user when the user wears the goggles 1. The annular pads 6 are soft and elastic to be well fitted to the face of the user and to cushion the physical shock.

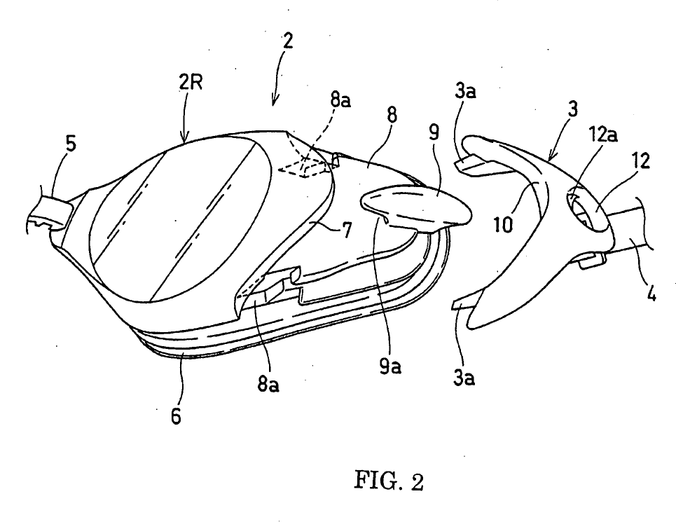

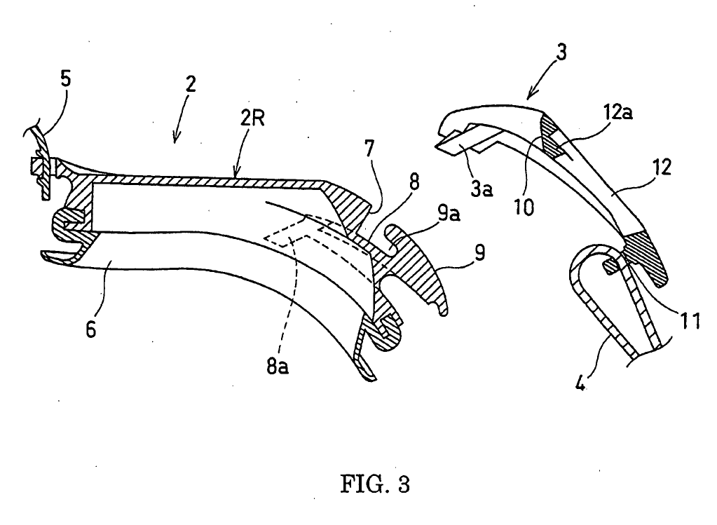

As shown in FIGS. 2 to 4, each of outer area of the main body 2 is provided with a wall 7 which is substantially perpendicular to a lens surface, and a pedestal 8 which extends outwardly from a base portion of the wall 7 to be substantially perpendicular to the wall 7. The pedestal 8, at its upper end lower ends, has fitting grooves 8a into which the later-described fitting portions 3a of the connecting tool 3 are fitted. The pedestal 8 is also provided at its outer portion with a protrusion 9 having a connecting groove 9a facing toward the wall 7. In the illustrated goggles, the wall 7 has a convex curved shape, and the pedestal 8 which extends outwardly from the base portion of the wall 7 is provided on its upper and lower ends with the fitting grooves 8a having inwardly inclining surfaces. The protrusion 9 is made to have a streamlined shape so as to reduce water resistance when a user wears the goggles 1.

The connecting tools 3 may be made of transparent, semitransparent or opaque hard material such as hard synthetic resin, or may be made of transparent, semitransparent or opaque soft material such as elastomer, and therefore the connecting tools 3 can be elastically deformable. The connecting tools 3 is provided with an inclined wall 10 which is inclined from front to back on the inner side thereof. The inclined wall 10 is provided at its opposite ends with fitting portions 4a which are fitted into the fitting grooves 8a of the pedestal 8. The connecting tools 3 is provided at the outer side thereof with a hooking portion 11 for connecting the attaching member 4, and also provided at the intermediate portion of the connecting tools 3 with a connecting hole 12 into which the protrusion 9 is inserted. The inner portion of the connecting hole 12 is provided with a connecting portion 12a being engaged with the connecting groove 9a of the protrusion 9. In the illustrated goggles, the inclined wall 10 has a recessed curved shape so as to fit to the wall 7, and fitting portions 4a having rhombic shape to be fitted with the fitting groove 8a are formed on opposite ends of the inclined wall 10. The connecting hole 12 is made to have a streamlined shape corresponding to the protrusion 9, so that water resistance can be reduced when a user wears the goggles 1. Further, since the fitting groove 8a of the main body 2 has an inclined surface inclining inwardly, and the fitting portion 3a of the connecting tool 3 has a rhombic shape corresponding to the fitting groove 8a, the fitting portion 4a is smoothly fitted into the fitting groove 8a.

It is preferable that the attaching member 4 is a band having elasticity for securing sufficient adhesion between a face of a user and the annular pad 6 of the main body 2 when the user wears the goggle 1, but a band having no elasticity or other member may be employed. That is, the attaching member 4 does not have to be a belt-like member such as a band, and can be of a rod-like member such as a temple. The both ends of the illustrated attaching member 4 are inserted through the insertion holes which are the hooking portions 11 of the connecting tools 3 and then folded back. The folded back belt portion is inserted through two parallel insertion holes (not shown) of a buckle 13 such that the length of the belt can be adjustable.

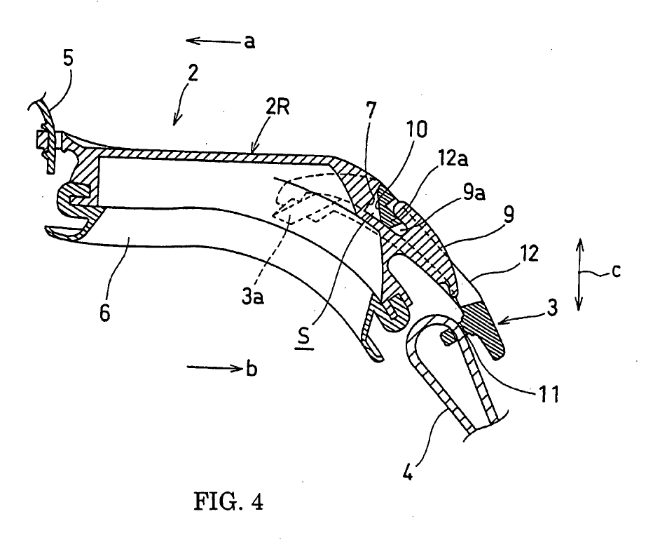

In order to connect the connecting tools 3 with the main body 2 of the goggles of the invention, the protrusion 9 projecting from the pedestal 8 of the main body 2 is inserted into the connecting hole 12 of the connecting tool 3, so that the connecting portion 12a of the connecting hole 12 is hooked in the connecting groove 9a of the protrusion 9, and the inclined wall 10 of the connecting tool 3 is abutted against the wall 7 of the main body 2. At this time, since the inclined wall 10 of the connecting tool 3 is inclined from front to back, only a front end of the inclined wall 10 is in contact with the wall 7 of the main body 2 to form a space S between the wall 7 and the inclined wall 10, as shown in FIG. 4. Further, since only the front end of the inclined wall 10 is in contact with the wall 7 of the main body 2 when the connecting portion 12a of the connecting hole 12 is hooked in the connecting groove 9a of the protrusion 9, the connecting tool 3 is smoothly connected to the main body 2. The abutment of the inclined wall 10 of the connecting tool 3 with the wall 7 of the main body 2 prevents the connecting tool 3 from disengaging from the main body 2 inwardly (in the direction of the arrow a in FIG. 4). Even when the connecting tool 3 is made of hard material, because of the engagement of the connecting groove 9a of the protrusion 9 and the connecting portion 12a of the connecting hole 12, the connecting tool 3 does not easily come off from the main body 2, and this engagement prevents the connecting tool 3 from disengaging from the main body 2 outwardly (in the direction of the arrow b in FIG. 4), forwardly and backwardly (in the both directions of the arrow c in FIG. 4), and upwardly and downwardly (in the both directions of the arrow d in FIG. 1).

The abutment of the inclined wall 10 of the connecting tool 3 with the wall 7 of the main body 2 allows the fitting grooves 8a formed in the pedestal 8 of the main body 2 and the fitting portions 4a formed on the both ends of the inclined wall 10 of the connecting tool 3 to be aligned with each other in position, and the fitting portions 4a are fitted into the fitting grooves 8a. The engagement of the fitting portions 3a with the fitting grooves 8a prevents the connecting tool 3 from disengaging from the main body 2 upwardly and downwardly (in the both directions of the arrow d in FIG. 1).

Consequently, according to the goggles of the present invention, the connecting tool 3 of the main body 2 and the attaching member 4 does not easily come off from the main body 2, even if it receives forces from any directions.

What is claimed is:

1. Goggles including a main body (2), connecting tools (3), and an attaching member (4) which is connected to outer ends of the main body (2) by the connecting tools (3);
wherein the main body (2) comprises at an outer area thereof:
walls 7 extending substantially perpendicularly to a lens surface;
pedestals (8) extending outwardly from base portions of the walls (7) to be substantially perpendicular to the walls 7; and
protrusions (9) formed on the pedestals (8) and provided with connecting grooves (9a) facing toward the walls (7); and wherein the connecting tools (3) comprise connecting holes (12) for the protrusions (9) which are provided with connecting portions (12a) being engaged with the connecting grooves (9a) of the protrusions (9) on inner sides of the connecting holes (12).

2. The goggles according to claim 1, wherein the connecting tool (3) further comprises an inclined wall (10) inclining from front to back on an inner side of the connecting tool (3), and a hooking portion 11 for the attaching member (4) on an outer side of the connecting tool (3).

3. The goggles according to claim 2, wherein the pedestal (8) is further provided at its upper and lower portions with fitting grooves (8a), and the inclined wall (10) is further provided at its opposite ends with fitting portions (3a) which are fitted into the fitting grooves (8a) of the pedestal (8).

4. The goggles according to claim 1, wherein the main body (2) comprises left and right bodies (2L, 2R) and a joint (5) connecting inner ends of the left and right bodies (2L, 2R).

5. The goggles according to claim 2, wherein the main body (2) comprises left and right bodies (2L, 2R) and a joint (5) connecting inner ends of the left and right bodies (2L, 2R).

6. The goggles according to claim 3, wherein the main body (2) comprises left and right bodies (2L, 2R) and a joint (5) connecting inner ends of the left and right bodies (2L, 2R).

7. The goggles according to claim 1, wherein the protrusions (9) of the main body (2) and the connecting holes (12) of the connecting tools (3) respectively have streamlined shapes.

8. The goggles according to claim 2, wherein the protrusions (9) of the main body (2) and the connecting holes (12) of the connecting tools (3) respectively have streamlined shapes.

9. The goggles according to claim 3, wherein the protrusions (9) of the main body (2) and the connecting holes (12) of the connecting tools (3) respectively have streamlined shapes.
